(19) United States
(12) Patent Application Publication
(10) Pub. No.: US 2009/0294688 A1
(43) Pub. Date: Dec. 3, 2009

(54) Ultraviolet Radiation Treatment System

(75) Inventor: Lionel Gordon Evans, Pukekohe (NZ)

Correspondence Address: JACOBSON HOLMAN PLLC, 400 SEVENTH STREET N.W., SUITE 600, WASHINGTON, DC, 20004, US

(73) Assignee: LOGISTIC SOLUTIONS LIMITED, Manuaku City (NZ)

(21) Appl. No.: 11/992,232

(22) PCT Filed: Sep. 14, 2006
(86) PCT No.: PCT/NZ2006/000239

§ 371 Date: May 11, 2009

(30) Foreign Application Priority Data
Sep. 20, 2005 (NZ) 542509

(51) Int. Cl. G01N 23/12

(52) U.S. Cl. 250/436

(57) ABSTRACT

Apparatus for treating a fluid with UV light which comprises or includes apparatus to confine a flow path of the fluid to be treated between an inner boundary defining tube (“inner tube”) transparent to the UV light and an outer boundary defining tube (“outer tube”) transparent to the UV light, and a UV light emitting device interiorly of the inner tube, and at least one UV light emitting device from outside of the outer tube.

### TECHNICAL FIELD

The present invention relates to a treatment system. In particular, a system capable of treating a substance via exposure to ultraviolet (UV) light (radiation).

### BACKGROUND ART

Ultraviolet (UV) light is defined as electro magnetic radiation having wavelengths shorter than visible light but longer than X-rays. The UV wavelength band is substantially 400100 nm. UV is usually divided into three components, with increasing energy, UV-A (320-400 nm), UV-B (280-320 nm) and UV-C (200-280 nm).

UV radiation is used in a number of existing applications such as in industrial coatings to provide scratch resistant finishes or pearl or metallic special effects, in advanced lithography in the manufacture of semiconductor circuit boards, in UV transilluminators for the imaging of UV fluorescent substances and in the disinfection of substances such as water from micro organisms as well as uses in beverage and sewage treatment.

UV is known to be highly lethal against bacteria, viruses, algae, moulds and yeast, and disease causing oocysts such as cryptosporidium where the UV inactivates the DNA of the micro organism. In fact there are no known micro-organisms which are UV resistant. Certain viruses such as hepatitis and Legionella pneumophila can survive for considerable periods of time in chlorine, a common chemical disinfectant, but are

eliminated by exposure to UV.

UV treatment offers many advantages in the treatment of microbial contaminants, over alternatives such as chemical or heat treatment. Most importantly, UV does not introduce any chemicals to the liquid or solid being treated, it produces no by-products, and it does not alter the taste, pH, or most other commonly measurable physical properties of the substance being treated.

UV treatment is also more cost effective than alternative disinfection treatments in terms of maintenance of equipment and other operating costs such as operator training and energy efficiency.

A disadvantage of UV-disinfection over other forms of disinfection such as filtration is that it has no impact on certain chemical contaminants such as heavy metals. Depending on the application UV-disinfection systems commonly combine a filtration system to remove both micro organism and chemical contaminants.

However UV oxidation is effective against some chemicals which photolyze on exposure to UV or in combination with a photoreactive additive such as hydrogen peroxide. Examples of applications of UV oxidation include treatment of N-nitrosodimethylamine (NDMA) from drinking water, treatment of low level pesticide and herbicide contamination in drinking water and treatment of 1,4-dioxane in industrial waste water.

UV lamps used in purification systems preferably produce UV-C or “germicidal UV” radiation at a wavelength of about 253.7 nm (254 nm nominal). This wavelength has an efficient kill rate for all micro organisms (greater than 99.9%). However this assumes that an optimum dose of UV is delivered to all micro organisms.

Factors affecting the UV dose are exposure time, UV emission output of the UV light emitting device, transmissibility of the medium to be disinfected and the temperature of the lamp wall.

Maximum wave emissions occur when the lamp wall temperature is regulated at about 42° C. If the lamps are subjected to fluctuations in temperature the efficiency of UV light emission can be reduced by more than 40%, dramatically reducing the kill rate.

Exposure time is dependant at least on the flow rate of the substance before the UV source and the distance of the substance to the UV emitting device. Transmissibility reflects the penetration of UV through a volume of substance; which is dependent on the colour and consistency of the substance, be it liquid, solid or gas.

Efficient UV exposure of a substance is a limiting factor with current UV disinfection systems. More efficient systems can result in decreased maintenance costs by reducing the number of rounds of disinfection needed, increasing flow rates and/or volumes of a substance that can be processed per unit time.

UV disinfection treatment is currently applied to drinking water purification, the beverage industry such as beer and fruit juices as an alternative to pasteurization, food processing such as cut and whole fruit and chicken meat processing to remove bacterial contaminants such as Salmonella, liquid and solid sewage for the removal of E. coli bacterial contaminants, air purification for use in air conditioning of public buildings and treatment of fats and greases in the exhaust from hoods over grills in fast food outlets.

The present invention is directed to all such uses as well as, for example, the wine industry.

All references, including any patents or patent applications cited in this specification are hereby incorporated by reference. No admission is made that any reference constitutes prior art. The discussion of the references states what their authors assert and the applicants reserve the right to challenge the accuracy and pertinence of the cited documents. It will be clearly understood that, although a number of prior art publications are referred to herein; this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e., that it will be taken to mean an inclusion of not only the listed components it directly references, but also other non-specified components or elements. This rationale will also be used when the term ‘comprised’ or ‘comprising’ is used in relation to one or more steps in a method or process. It is an object of the present invention to provide an efficient UV purification system or at least to provide the public with a useful choice.

For the purposes of the specification the term “substance” or grammatical variations thereof may refer to any: gas, liquid or solid; which it is desired to treat with UV light.

For the purposes of the specification the term “receptacle” or grammatical variations thereof may refer to a conduit, container, or similar.

For the purposes of the specification the term “UV light transmissible material” or grammatical variations thereof may refer to any material capable of allowing the transmission of UV light. In particular, UV light transmissible material may include but should not be limited to: glass, plastic, fluoro-polymer and quartz.

As used herein the term “and/or” means “and” or “or”, or both.

As used herein the term “(s)” following a noun includes, as might be appropriate, the singular or plural forms of that noun.

Further aspects and advantages of the present invention will become apparent from the ensuing description which is given by way of example only.

### DISCLOSURE OF INVENTION

In an aspect the invention is method of treating a fluid which comprises or includes

confining a flow path of the fluid between an inner boundary defining tube (“inner tube”) transparent to UV light and an outer boundary defining tube (“inner tube” or “outer tube”) transparent to UV light, and

irradiating the fluid passing between said boundaries with both UV light from a UV light emitting device interiorly of the inner tube and with UV light from at least one UV light emitting device from outside of the exterior tube.

Preferably there is an array of plural UV light emitting devices about the outer tube.

Ideally said inner tube is of circular transverse surfaces and said outer tube is of circular transverse surfaces.

Preferably said inner and outer tubes are aligned with an in common or parallel longitudinally axis.

Preferably a rotating flow of the fluid to be treated is caused between the inner and outer tubes as the fluid moves along and between the tubes. We have found that the spinning motion has increased the “microbial kill” by 1.5 to 2 log.

Preferably the flow path is of a dropping fluid.

Preferably said tubes are aligned substantially vertically.

In the most preferred form a rotating flow of the fluid to be treated is caused between the inner and outer tubes by at least an in feed of the fluid substantially as a tangential flow to one or other of the tubes and/or the annular space there between. Preferably there is a complementary out feed of a tangential type from below the tubes.

Preferably the light is UVc light.

Preferably at least one tube (most preferably both tubes) is of an extruded fluorinated ethylene propylene (FEP).

Preferably at least one or more of the UV light emitting devices is maintained at a surface temperature of about 42° C.

Preferably the UV light from at least one or more of the UV light emitting devices is at a wave length of in the range from 250 to 260 nm.

Most preferably the UV light emitting devices each has a wave length of substantially 253.7 nm (nominally 254 nm).

Preferably an air or gas flow is induced passed the UV light device in said inner tube.

Preferably an air or gas flow is induced passed the UV light device(s) about said outer tube.

Preferably reflectors reflect UV light that otherwise would not enter the outer tube from the UV light device(s) about the outer tube directly and/or indirectly to the outer tube.

Preferably reflectors (or other members) baffle at least in part direct UV irradiation from one UV light device about the outer tube to another such tube.

Preferably the fluid is a liquid or includes a liquid carrier (e.g. wine or a wine precursor).

In another aspect the invention is apparatus for treating a fluid with UV light which comprises or includes

apparatus to confine a flow path of the fluid to be treated between an inner boundary defining tube (“inner tube”) transparent to the UV light and an outer boundary defining tube (“outer tube”) transparent to the UV light, and

a UV light emitting device interiorly of the inner tube, and

at least one UV light emitting device from outside of the outer tube.

Preferably there is an array of plural UV light emitting devices about the outer tube.

Preferably said inner tube is of circular transverse surfaces.

Preferably said outer tube is of circular transverse surfaces.

Preferably said inner and outer tubes are aligned with an in common or parallel longitudinally axis.

Ideally the inlet and outlet is such that a rotating flow of the fluid to be treated is caused between the inner and outer tubes as the fluid moves along and between the tubes.

We have found that the spinning motion has increased the “microbial kill” by 1.5 to 2 log.

Preferably the flow path is of a dropping fluid.

Preferably said tubes are aligned substantially vertically.

Most preferably a rotating flow of the fluid to be treated is caused between the inner and outer tubes by at least an in feed of the fluid substantially as a tangential flow to one or other of the tubes and/or the annular space there between. Preferably there is a complementary out feed of a tangential type from below the tubes.

Preferably the light is UVc light.

Preferably at least one tube is of an extruded fluorinated ethylene propylene (FEP). Preferably both said tubes are of FEP.

Preferably at least one or more of the UV light emitting devices is maintained at a surface temperature of about 42° C.

Preferably the UV light from at least one or more of the UV light emitting devices is at a wave length in the range of from 250 to 260 nm.

Preferably the UV light emitting devices each has a wave length of substantially 253.7 nm (nominally 254 nm).

Preferably there is apparatus to provide an air or gas flow passed the UV light device in said inner tube.

Preferably there is apparatus to provide an air or gas flow passed the UV light device(s) about said outer tube.

Preferably there are reflectors to reflect UV light that otherwise would not enter the outer tube from the UV light device(s) about the outer tube directly and/or indirectly to the outer tube.

Preferably reflectors or other members baffle at least in part direct UV irradiation from one UV light device about the outer tube to another such tube.

In yet another aspect the invention is a method of treating wine or a wine precursor, said method comprising or including the steps of

feeding the wine downwardly in a helical flow between an inner and an outer tube transparent to UV light, and

irradiating the wine or wine precursor between said tubes with UV light from UV light emitting devices from both interiorly of the inner tube and from exteriorly of the outer tube.

We have found that the spinning motion has increased the “microbial kill” by 1.5 to 2 log.

Preferably the UV light is at a wave length of substantially 254 nm.

Preferably there is an array of multiple UV light emitting devices about the outer tube.

Preferably both tubes are of FEP.

Preferably the surface temperature of the UV light emitting devices is maintained at a substantially constant temperature.

Preferably said substantially constant temperature is about 42° C.

Preferably the light is UVc light.

Preferably reflectors reflect and/or baffle at least in part UV light from multiple UV light emitting devices to better direct light from each UV light emitting device to the outer tube and/or away from other UV light emitting devices.

In another aspect the invention is a substance or fluid treated by a method of the present invention or by a system of the present invention.

According to another aspect of the present invention there is provided a UV treatment system which includes:
at least one UV light emitting device
a receptacle made from a UV light transmissible material in which a substance to be treated can be located, the system being characterised in that the receptacle is configured to include a channel therein such that the receptacle has an outer peripheral surface on the outside thereof and an inner peripheral surface where the channel is located;
wherein there is at least one UV light emitting device positioned so as to be capable of exposing at least a substantial portion of at least one region of the outer peripheral surface of the receptacle to UV light;

and wherein there is also provided at least one further UV light emitting device positioned within the channel of the receptacle capable of exposing at least a substantial portion of at least one region of the inner peripheral surface of the channel to UV light.

A treatment system with this configuration increases the surface area of the receptacle and thus enhancing the ability of the UV radiation to penetrate the substance to be treated.

The UV treatment may be any process that is facilitated by exposure of the substance to UV light. For example, the treatment can include but should not be limited to antimicrobial, sterilization, DNA disruption and oxidising type applications. However, this list should not be seen as limiting.

The UV light emitting device may be any light emitting device capable of producing light (i.e. electromagnetic radiation) at a wave length within the broad ultra violet range of substantially 400-100 nm.

In some embodiments the UV light emitting device may be in the form of at least one LED.

In a preferred embodiment the UV light emitting device may be in the form of a UV lamp such as a tungsten-halogen lamp.

In a preferred embodiment the UV lamp, bulb or tube may be coated with a fluoropolymer material such as polytetrafluoroethylene (PTFE).

Preferably, the UV light emitting device may a UV lamp capable of emitting a nominal wavelength of substantially 254 nm.

Most preferably, the UV light emitting device may be a UV lamp emitting a wavelength of substantially 253.7 nm.

In a preferred embodiment, the emission output of the UV emitting device may be regulated to provide a variable level of UV radiation.

In a more preferred embodiment, the emission output of the UV emitting device may be regulated with an electronic device such as a rheostat.

The UV emitting device(s) may be positioned about the receptacle in a variety of different configurations.

In some embodiments where the UV emitting device is in the form of a single LED the LED may be shaped so as to substantially surround the outer peripheral surface of the receptacle. For example the LED may have substantially circular or semi-circular cross sectional shape.

In a preferred embodiment the UV treatment system includes eight UV lamps arranged uniformly around the outer peripheral surface of the receptacle.

In a preferred embodiment the further UV light emitting device may be a UV lamp positioned centrally within the channel of the receptacle.

In a preferred embodiment the further UV lamp may be positioned within a smaller diameter tube.

In a preferred embodiment the receptacle may be a tube with a high UV transmission.

In a more preferred embodiment the receptacle may be a tube with a UV transmission in excess of 85%.

In a more preferred embodiment the receptacle may be a tube of extruded fluorinated ethylene propylene (FEP). FEP is also known as advanced fluoropolymer (AFP).

In a preferred embodiment the smaller diameter tube may be made of a material with a high UV transmission.

In a more preferred embodiment the smaller diameter tube may be made of extruded FEP.

In a preferred embodiment the UV lamps have a regulated wall temperature of 42° C.

In a more preferred embodiment a thermostatically controlled air fan may be used to maintain the lamp wall temperature.

In a preferred embodiment the receptacle is a conduit through which liquid flows from an inlet through the UV treatment system to an outlet.

In a more preferred embodiment the incoming liquid flow may be directed through a spiral that imparts a spinning motion to the liquid as it flows through the receptacle.

In a preferred embodiment the UV treatment system is used to disinfect a liquid selected from the group consisting of: wine, blood, beer, water, sewage.

Thus preferred embodiments of the present invention may have a number of advantages over the prior art which include more efficient treatment of a substance by exposure to more UV light.

### BRIEF DESCRIPTION OF DRAWINGS

Further aspects of the present invention will become apparent from the following description which is given by way of example only and with reference to the accompanying drawings in which:

FIG. 1 shows a side elevation view of one embodiment of a UV treatment system of the present invention;

FIG. 2 shows a side elevation view of the embodiment shown in FIG. 1;

FIG. 3 shows a section view of the embodiment shown in FIG. 1,

FIG. 4 shows a cross section schematic view of the embodiment shown in FIG. 1,

FIG. 5 is a plan view from above and/or below of an array of baffles interposed between an array, by way of example, of eight UV generating tubes disposed equi-distantly around the outer tube, the annular space in which the flow path is to move helically being shown in as a solid block,

FIG. 6 is a similar view to that of FIG. 5 but showing reflector plates disposed outwardly of each UV tube so as to better confine and reflect the light from such tubes back towards the outer tube of the flow path (directly or via reflector baffles),

FIG. 7 is a perspective view of one end attachment of an arrangement that can be used input and cause, or take out, a helical flow, for the sake of clarification, FIG. 7 showing an inlet which introduces into a blocked tube a helical flow of a fluid to be treated, the outer UV transparent or translucent (“transparent”) tube of the apparatus being shown in broken lines and the break in the length of the tube showing how, at any stage, the UV transparent or translucent tube can be connected,

FIG. 8 is another elevational view of the arrangement of FIG. 7,

FIG. 9 is yet another elevational arrangement of the perspective view of FIG. 7, and

FIG. 10 is a plan view from above of the arrangement shown in FIG. 7 through 9.

### BEST MODES FOR CARRYING OUT THE INVENTION

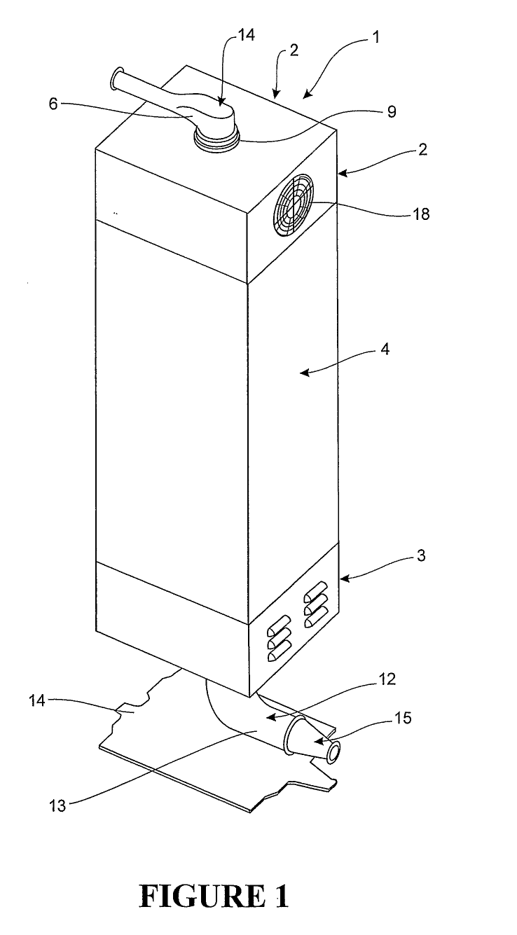

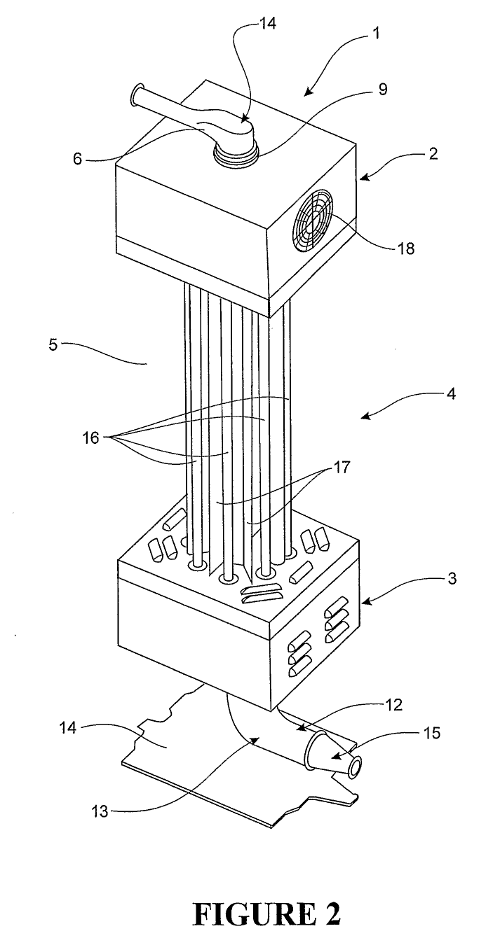

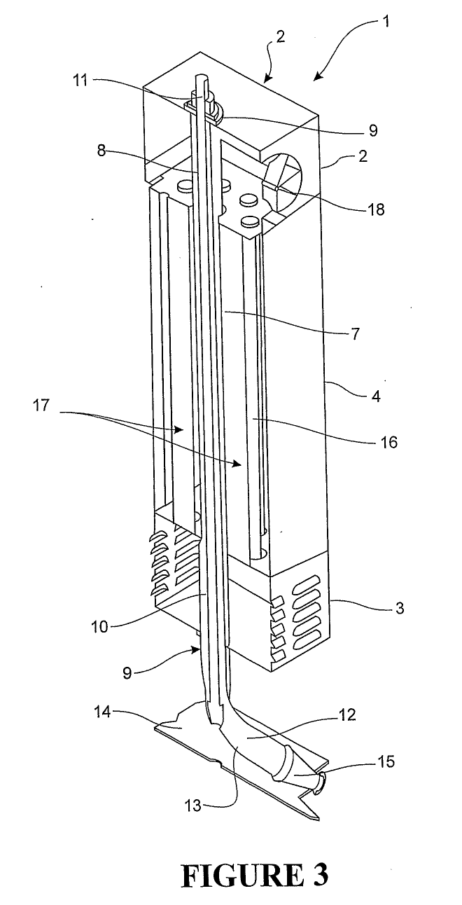

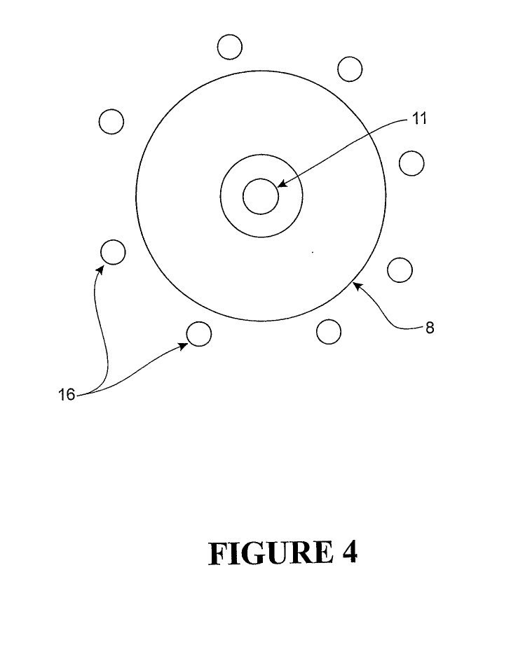

The invention is now described in relation to one preferred embodiment of the present invention as shown in FIGS. 1 to 6. It should be appreciated that the invention may be varied from the Figures without departing from the scope of the invention.

Referring to FIGS. 1 to 3, a UV treatment system is shown generally indicated by arrow 1. The UV treatment system has a top lid 2 and a bottom lid 3 enclosing the top and bottom ends of the UV treatment system respectively, and an enclosure box 4, enclosing the central portion of the UV treatment system.

The top lid 2, bottom lid 3 and enclosure box 4 may be fabricated in stainless steel as it provides a hygienic non-porous surface, although this should not be seen as limiting as other materials may be used as appropriate to the environment for use.

The enclosure box 4 has a backing plate 5 which has a number of apertures (not shown) which facilitate the fixing of the UV treatment system to a support (not shown).

In use a substance such as a liquid enters the top of the UV treatment system through a tri flow connector 6 which imparts a spinning motion to the liquid as it flows through a receptacle in the form of a conduit 7 having a central channel 8.

The spinning flow characteristics of the UV treatment system increase the potential UV treatment capability.

The tri flow connector 6 is connected to the conduit 7 by a top connector 8 which is attached to the top lid 2 at a pan screw head 9.

The liquid exits the conduit 7 and passes into a bottom connector 10 which is attached to the bottom lid 3 at a pan screw head 9.

A smaller diameter UV transmissible tube 11, which houses an additional UV light emitting device, is positioned centrally within the conduit 7.

The additional UV emitting device may be a UV fluorescent tube.

The liquid exits the UV treatment system via a seal pipe 12, which incorporates an outlet bend 13 and an access port 14 to the smaller diameter tube 11.

The seal pipe 12 is reduced in diameter by an outer reducer 15.

The tri flow connector 6 also incorporates an access port 14 to the smaller diameter tube

The full length of the conduit 7 is vertically surrounded by a symmetrical arrangement of UV lamps 16.

The length of the conduit 7 is determined by the required treatment time.

The UV lamps 16 are partitioned from each other by dividers 17.

The top lid 2 incorporates a thermostatically controlled air fan which is used to maintain the UV lamp wall temperature.

The bottom lid 3 incorporates a vent 19 to allow air to be drawn into the UV treatment system via the fan 18.

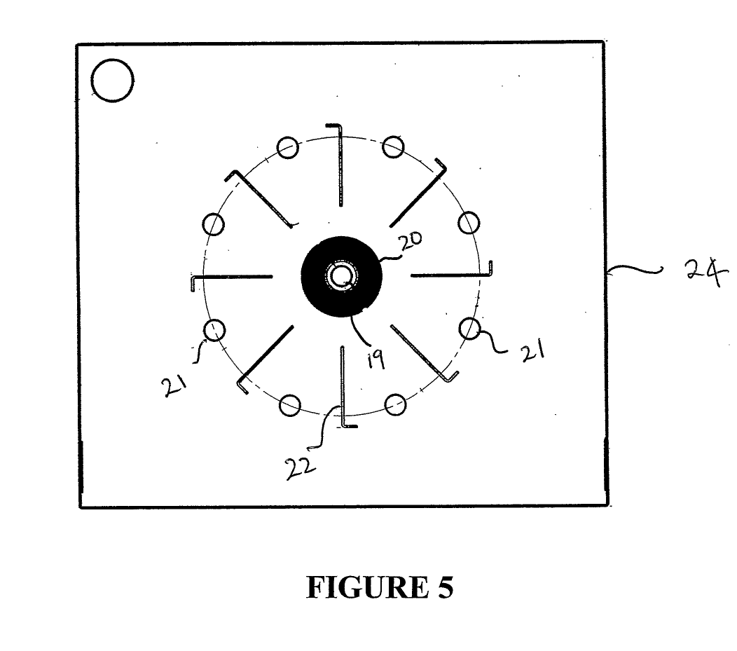

By way of example, FIGS. 5 through 10 show a preferred array.

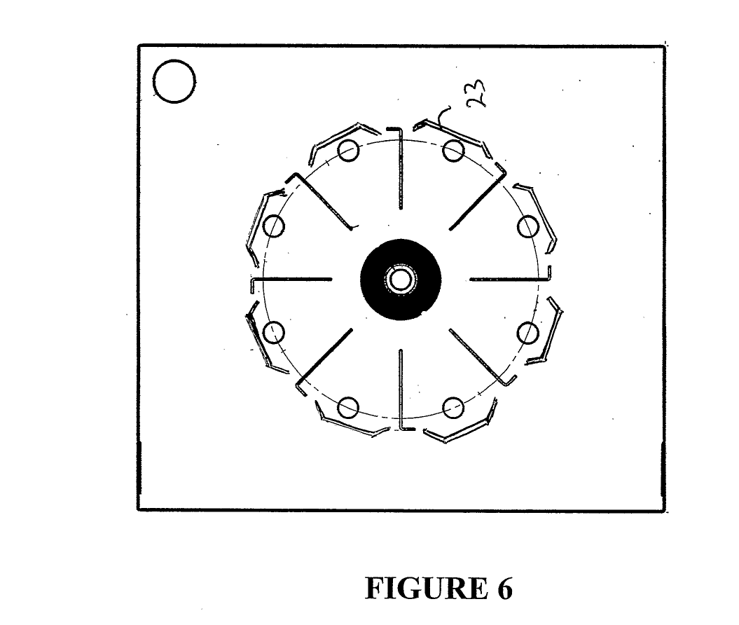

By way of example, and in no way limiting, the arrangement as shown in FIGS. 5 and 6 shows an inner tube to generate UVc light 19 which is interiorly of the annular space 20 downwards which the liquid or fluid to be treated is the flow. Arrayed around that are, by way of example eight UVc tubes 21 and each is masked from its neighbour by a baffle (preferably reflectors of SS) 22. These baffles (preferably alternate baffles) may optionally include openings but at positions therein that preferably prevent incident light from one tube 21 reaching any neighbouring tube 21 directly.

Disposed about the array of tubes 21 are reflectors 23 (e.g. of SS) and they function to direct UV light that would otherwise be wasted back towards the outer tube and thus the fluid 20 via the outer FEC tube either directly or via a bouncing off of one or more baffles 22.

Preferably the array as depicted is contained within a chamber as previously described, the chamber having boundaries 24 as shown in FIG. 5. Preferably there is an air or gas flow so as to maintain cooling in that chamber of the tubes 21 and 19.

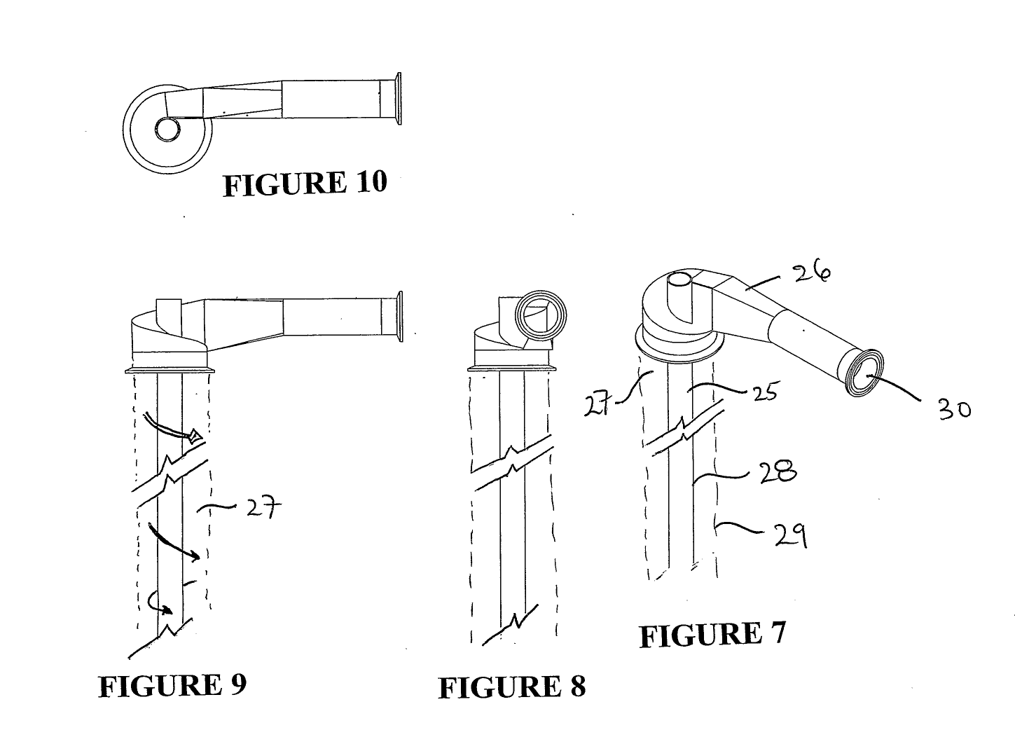

The arrangement as shown in FIGS. 7 through 10 shows for the top of the flow path (but its complement inverted can be used for the lower end) a tubular portion 25 (e.g. stainless steel) which provides the structure about which the helical arrangement 26 provides a tangential feed into the annular space 27. This is between the inner UV transparent tube 28 and the outer UV transparent tube 29 shown in broken outline. Preferably the inner tube 28 is to connect to the tube 25 in some appropriate way.

As can be seen, a feed in via the inlet 30 will have the effect of starting a helical flow in the space 27 as shown in the elevational view of FIG. 9.

By way of example, a preferred arrangement has an inner UV transparent tube of diameter 26 mm and an outer UV transparent tube of 60 mm diameter thus defining the space 27. This can be of any appropriate length to ensure the appropriate treatment outcome results taking into account the nature of the material to be treated, the intensity and nature of the lighting, the speed of the through put, the thickness of the FEP or other material through which irradiation is to occur, etc.

By way of example the spacing from one end to another of the baffles can be of, for example, 228 mm and each radially extends inwardly about 61 mm.

A suitable lamp to generate UVc light in each instance is one of 75 watts with an output of 23 watts UVc radiation. Preferably such tubes are medium pressure mercury vapour lamps best able to generate UVc radiation of nominally 254 nm at a lamp skin temperature at or near 42° C.

Such an arrangement has been found on a wine throughput on, for example, E. coli bacteria to be an effective 99.99% control using when 6600 μWs/cm² and, in respect of the yeast Saccharomyces Wilianus, to be an effective 99.99% control with 37800 μWs/cm².

We have found that the spinning motion has increased the “microbial kill” by 1.5 to 2 log.

We believe therefore, with the eight peripheral lamps described each of 75 watts power requirement plus a central lamp of 75 watts power requirement, and with an FEP wall thickness of 1 mm for the internal tube and an FEP wall thickness of about 0.5 mm for the outer tube, it is possible to get up to 450000 μWs/cm² going into the walls of the tubes. With the expected transmission of such material, levels sufficient to kill of bacteria and yeast typified by that previously described are obtained with such a geometry.

Aspects of the present invention have been described by way of example only and it should be appreciated that modifications and additions may be made thereto without departing from the scope thereof.

1. A method of treating a fluid which comprises or includes
confining a flow path of the fluid between an inner boundary defining tube (“inner tube”) transparent to UV light and an outer boundary defining tube (“inner tube” or “outer tube”) transparent to UV light, and
irradiating the fluid passing between said boundaries with both UV light from a UV light emitting device interiorly of the inner tube and with UV light from at least one UV light emitting device from outside of the exterior tube.

2. The method of claim 1 wherein there is an array of plural UV light emitting devices about the outer tube.

3. The method as claimed in claim 1 wherein said inner tube is of circular transverse surfaces.

4. The method of claim 1 wherein said outer tube is of circular transverse surfaces.

5. The method as claimed in claim 3 wherein said inner and outer tubes are aligned with an in common or parallel longitudinally axis.

6. The method as claimed in claim 5 wherein a rotating flow of the fluid to be treated is caused between the inner and outer tubes as the fluid moves along and between the tubes.

7. The method as claimed in claim 1 wherein the flow path is of a dropping fluid.

8. The method as claimed in claim 7 wherein said tubes are aligned substantially vertically.

9. The method as claimed in claim 8 wherein a rotating flow of the fluid to be treated is caused between the inner and outer tubes by at least an in feed of the fluid substantially as a tangential flow to one or other of the tubes and/or the annular space there between.

10. The method as claimed in claim 9 wherein there is a complementary out feed of a tangential type from below the tubes.

11. The method as claimed in claim 1 wherein the light is UVC light.

12. The method as claimed in claim 1 wherein at least one tube is of an extruded fluorinated ethylene propylene (FEP).

13. The method as claimed in claim 1 wherein both said tubes are of FEP.

14. The method as claimed in claim 1 wherein at least one or more of the UV light emitting devices is maintained at a surface temperature of about 42° C.

15. The method as claimed in claim 1 wherein the UV light from at least one or more of the UV light emitting devices is at a wave length of in the range from 250 to 260 nm.

16. The method as claimed in claim 1 wherein the UV light emitting devices each has a wave length of substantially 253.7 nm.

17. The method as claimed in claim 1 wherein an air or gas flow is induced passed the UV light device in said inner tube.

18. The method as claimed in claim 1 wherein an air or gas flow is induced passed the UV light device(s) about said outer tube.

19. The method as claimed in claim 1 wherein reflectors reflect UV light that otherwise would not enter the outer tube from the UV light device(s) about the outer tube directly and/or indirectly to the outer tube.

20. The method as claimed in claim 15 wherein reflectors baffle at least in part direct UV irradiation from one UV light device about the outer tube to another such tube.

21. The method of claim 1 wherein the fluid is a liquid or includes a liquid carrier.

22. The method of claim 21 wherein the fluid is wine or a wine precursor.

23. An apparatus for treating a fluid with UV light which comprises or includes
apparatus to confine a flow path of the fluid to be treated between an inner boundary defining tube (“inner tube”) transparent to the UV light and an outer boundary defining tube (“outer tube”) transparent to the UV light, and
a UV light emitting device interiorly of the inner tube, and
at least one UV light emitting device from outside of the outer tube.

24. The apparatus of claim 23 wherein there is an array of plural UV light emitting devices about the outer tube.

25. The apparatus of claim 23 wherein said inner tube is of circular transverse surfaces.

26. The apparatus of claim 23 wherein said outer tube is of circular transverse surfaces.

27. The apparatus of claim 26 wherein said inner and outer tubes are aligned with an in common or parallel longitudinally axis.

28. The apparatus of claim 27 wherein the inlet and outlet is such that a rotating flow of the fluid to be treated is caused between the inner and outer tubes as the fluid moves along and between the tubes.

29. The apparatus of claim 23 wherein the flow path is of a dropping fluid.

30. The apparatus of claim 29 wherein said tubes are aligned substantially vertically.

31. The apparatus of claim 30 wherein a rotating flow of the fluid to be treated is caused between the inner and outer tubes by at least an in feed of the fluid substantially as a tangential flow to one or other of the tubes and/or the annular space there between.

32. The apparatus of claim 31 wherein there is a complementary out feed of a tangential type from below the tubes.

33. The apparatus of claim 23 wherein the light is UVc light.

34. The apparatus of claim 23 wherein at least one tube is of an extruded fluorinated ethylene propylene (FEP).

35. The apparatus of claim 34 wherein both said tubes are of FEP.

36. The apparatus of claim 23 wherein at least one or more of the UV light emitting devices is maintained at a surface temperature of about 42° C.

37. The apparatus of claim 23 wherein the UV light from at least one or more of the UV light emitting devices is at a wave length in the range of from 250 to 260 nm.

38. The apparatus of claim 23 wherein the UV light emitting devices each has a wave length of substantially 253.7 nm.

39. The apparatus of claim 23 where there is apparatus to provide an air or gas flow passed the UV light device in said inner tube.

40. The apparatus of claim 23 wherein there is apparatus to provide an air or gas flow passed the UV light device(s) about said outer tube.

41. The apparatus of claim 23 wherein there are reflectors to reflect UV light that otherwise would not enter the outer tube from the UV light device(s) about the outer tube directly and/or indirectly to the outer tube.

42. The apparatus of claim 41 wherein reflectors baffle at least in part direct UV irradiation from one UV light device about the outer tube to another such tube.

43. A method of treating wine or a wine precursor, said method comprising or including the steps of
feeding the wine downwardly in a helical flow between an inner and an outer tube transparent to UV light, and
irradiating the wine or wine precursor between said tubes with UV light from UV light emitting devices from both interiorly of the inner tube and from exteriorly of the outer tube.

44. The method of claim 43 UV light is at a wave length of substantially 254 nm.

45. The method as claimed in claim 43 wherein there is an array of multiple UV light emitting devices about the outer tube.

46. The method as claimed in claim 43 wherein both tubes are of FEP.

47. The method as claimed in claim 43 wherein the surface temperature of the UV light emitting devices is maintained at a substantially constant temperature.

48. The method as claimed in claim 47 wherein said substantially constant temperature is about 42° C.

49. The method as claimed in claim 43 wherein the light is UVc light.

50. The method as claimed in claim 43 wherein reflectors reflect and/or baffle at least in part UV light from multiple UV light emitting devices to better direct light from each UV light emitting device to the outer tube and/or away from other UV light emitting devices.

51. A UV treatment system which includes:
at least one UV light emitting device
a receptacle made from a UV light transmissible material in which a substance to be treated can be located, the system being characterised in that the receptacle is configured to include a channel therein such that the receptacle has an outer peripheral surface on the outside thereof and an inner peripheral surface where the channel is located;
wherein there is at least one UV light emitting device positioned so as to be capable of exposing at least a substantial portion of at least one region of the outer peripheral surface of the receptacle to UV light;
and wherein there is also provided at least one further UV light emitting device positioned within the channel of the receptacle capable of exposing at least a substantial portion of at least one region of the inner peripheral surface of the channel to UV light.

52. (canceled)
